(19) United States
(12) Patent Application Publication
(10) Pub. No.: US 2002/0082357 A1
(43) Pub. Date: Jun. 27, 2002

(54) Resin and its use in converting morphine to codeine

(76) Inventor: Robert C. Corcoran, Tie Siding, WY (US)

Correspondence Address: FINNEGAN, HENDERSON, FARABOW,, GARRETT & DUNNER, L.L.P., 1300 I STREET, N.W., WASHINGTON, DC, 20005-3315, US

(21) Appl. No.: 09/970,860

(22) Filed: Oct. 5, 2001

Related U.S. Application Data
(60) Provisional application No. 60/238,697, filed on Oct. 6, 2000.

(51) Int. Cl. C08F 8/34; C07D 498/06

(52) U.S. Cl. 525/333.5; 546/44

(57) ABSTRACT

A resin and its use as a methylating agent. One embodiment is a resin comprising a solid support and cationic methylated sulfonium, sulfoxonium, selenonium or phosphonium salts immobilized on the solid support. Another embodiment is the use of the resin as a methylating agent, for example in the conversion of morphine to codeine.

### CROSS REFERENCE TO RELATED APPLICATIONS

This patent application claims the benefit of priority under 35 U.S.C. § 119(e) to U.S. provisional application No. 60/238,697, filed on Oct. 6, 2000.

### FIELD OF THE INVENTION

The invention relates to a resin and its use as a methylating agent. One embodiment of the invention is a resin comprising a solid support and at least one cationic methylated sulfonium, sulfoxonium, selenonium or phosphonium salt immobilized on the solid support. Another embodiment of the invention is the use of the resin as a methylating agent, for example in the conversion of morphine to codeine.

### BACKGROUND

Methylation of the phenolic hydroxyl group of morphine produces codeine. Use of conventional methylating agents for that conversion, such as iodomethane and dimethylsulfate, suffers from the problem that the methylation may occur competitively at the nitrogen of the morphine molecule to give a quaternary ammonium salt. One method for avoiding this problem involves reacting morphine with a trimethylanilinium salt, for example in the salt's hydroxide form, to give the trimethylanilinium phenolate salt of morphine. Heating this salt leads to methylation of the phenolate with concomitant formation of dimethylaniline.

Another method for methylating morphine to form codeine is discussed in U.S. Pat. No. 5,981,750. The '750 patent discloses attachment of a methyl (dialkyl or diaryl) anilinium group on a support to give a methylation resin. When in the hydroxide form, the methylation resin serves initially as an ion exchange resin, separating morphine and other phenolic compounds from a methanolic or ethanolic sample mixture. Replacement of the alcohol solvent with toluene or other non-polar solvent, followed by heating, results in the methylation reaction and release of the newly-formed codeine from the resin. In total, the method serves to both purify morphine and convert it to codeine. The expended methylation resin may then be converted to a dimethylaniline derivative, which can be re-methylated using dimethylsulfate or other alkylating agents. Subsequent conversion to the hydroxide or methoxide form gives regenerated methylation resin for re-use in morphine purification and conversion to codeine.

Although the methylation resin of the '750 patent offers improvements over methods using simple trimethylanilinium salts, it also carries some disadvantages. The disadvantages stem from the fact that trimethylanilinium salts are relatively poor methylating agents. For the methylation reaction to proceed at a useful rate, the solvent is changed from a polar protic solvent, such as methanol, used to load the sample on the methylation resin, to a non-polar aprotic solvent, such as toluene. In fact, the use of polar protic solvents in the reaction, such as water or alcohols, could reduce the rate of reaction to an impractical level. The loaded resin is then heated in the non-polar aprotic solvent at a high temperature, for example over 100° C., for about nine hours.

The changing of the solvent in the process of the '750 patent and the severity of the heating conditions both carry significant costs. The economic viability of industrial processes using large quantities of solvents depends in some part on the recovery and re-use those solvents. The process of the '750 patent produces methanol/toluene mixtures from the change of methanol to toluene. Solvent mixtures are also formed when the resin is regenerated for reuse. The recovery of solvents from the mixtures is less convenient than the recovery of a single solvent from non-volatile substances. In the former case a fractional distillation is used, while in the latter case a simple evaporation can be used. Thus, recovery of solvents from the process of the '750 patent may, in some instances, be less convenient, and the expense can offset economic advantages of solvent re-use.

In the methylation reaction of the '750 patent, the heating conditions in toluene can raise energy costs and affect worker safety. Toluene has a boiling point of 110° C.; requiring substantial energy resources to bring large amounts of the solvent to the requisite temperature on an industrial scale. These energy costs are then repeated during the solvent recovery process. The high temperatures involved in both the methylation and solvent recovery operations may pose safety issues for workers, and add costs to ensure a safe work environment.

### SUMMARY OF THE INVENTION

This invention relates to a novel resin and its use as a methylating agent. One embodiment of the invention relates to a resin comprising a solid support and at least one cationic methylated sulfonium, sulfoxonium, selenonium or phosphonium salt immobilized on the solid support, in which the counterion of the at least one cationic methylated salt is an anion that has a conjugate acid having a pKₐ of 10 or higher. Another embodiment of the invention relates to the use of the resin as a methylating agent, for example in the conversion of morphine to codeine.

The novel resin of the invention allows, in one embodiment, for the use of a single solvent in the methylation of phenolic compounds, such as morphine, to their corresponding methyl ethers, such as codeine, and regeneration of the methylation resin. Use of the single solvent provides a more convenient and less expensive means of solvent recovery and re-use compared to processes that use or result in solvent mixtures. The characteristics of the novel resin of the invention also permit for the use of a wider variety of solvents, including polar protic solvents, during the methylation reaction. These sulfonium, sulfoxonium, selenonium, and phosphonium salts, for example, can have enhanced nucleophilic and leaving group properties compared to nitrogen-based resins. The resin of the invention also allows, in one embodiment, for the use of less extreme heating conditions, such as lower heating temperatures and faster reaction times, than those disclosed for nitrogen-based resins.

Other embodiments of the invention relate to the novel resin in its hydroxide or alkoxide form, and resins where the at least one cationic methylated salt comprises a linker group between the sulfonium, sulfoxonium, selenonium or phosphonium atom of the salt and the solid support. Still other embodiments relate to the loading of morphine onto the resins in the presence of an alcohol, such as methanol, heating the loaded resin in the presence of the alcohol to form codeine, and regenerating the resin in the presence of the alcohol. Still other embodiments relate to the use of the resin in the alkylation of carboxylic acids and thiols.

### BRIEF DESCRIPTION OF THE DRAWINGS

FIG. 1 illustrates one possible synthesis of an example methylsulfonium resin of the invention.

FIGS. 2 and 3 illustrate some syntheses of a variety of methylsulfonium methylation resins of the invention.

FIG. 4 illustrates a number of example methylsulfonium resins according to the invention, which differ in the nature of substituents in the salts and the linker groups within the salts that immobilize them to a solid support.

FIG. 5 illustrates a number of example methylselenonium resins according to the invention, which differ in the nature of substituents in the salts and the linker groups within the salts that immobilize them to a solid support.

FIG. 6 illustrates a number of example methylphosphonium resins according to the invention, which differ in the nature of substituents in the salts and the linker groups within the salts that immobilize them to a solid support.

FIG. 7 illustrates a number of example methylsulfoxonium resins according to the invention, which differ in the nature of substituents in the salts and the linker groups within the salts that immobilize them to a solid support.

FIG. 8 illustrates the conversion of morphine to codeine in one embodiment of the invention, and regeneration of the expended resin in one embodiment of the invention.

### DETAILED DESCRIPTION OF THE INVENTION

One embodiment of the invention is a resin that comprises:

a solid support, and

at least one cationic methylated sulfonium, sulfoxonium or selenonium salt immobilized on the solid support,

in which the counterion of the at least one cationic methylated salt is an anion that has a conjugate acid having a pKₐ of 10 or higher.

Another embodiment of the invention is a resin that comprises:

a solid support, and

at least one cationic methylated phosphonium salt immobilized on the solid support,

in which the counterion of the at least one cationic methylated salt is an anion that has a conjugate acid having a pKₐ of 10 or higher.

The solid support of the resins may comprise, for example, one or more polymers. Such polymers include, for instance, homopolymers, co-polymers, blends of polymers, and combinations thereof. The solid support could comprise macroreticular resins or non-macroreticular polymeric supports. For example, the solid support may comprise polystyrene. The solid support may comprise, or be composed entirely of, materials other than polymers. For example, the solid support may comprise silica gel, alumina, or diatomaceous earth. The solid support can be chosen to exhibit stability to solvents, such as methanol, at temperatures that may be employed during use of the resin. The solid support can also be chosen to exhibit mechanical stability, as well as a moderate level of swelling upon any change of solvent types in its environment, for example from exposure to polar solvents to exposure to non-polar solvents.

The resins of the invention may comprise one or more cationic methylated sulfonium salts, or one or more cationic methylated sulfoxonium salts, or one or more cationic methylated selenonium salts, or one or more cationic methylated phosphonium salts, or any combination thereof, immobilized on the solid support. The cationic methylated salts may be “immobilized” on the solid support, for example, through a covalent bond, through dipolar attraction, or through ion pairing. The ion pairing may take place, for example, between a sulfate or sulfonate anion and a quaternary ammonium cation.

The counterion of the at least one cationic methylated salt is an anion that has a conjugate acid having a pKₐ of 10 or higher. For example, the anion can have a conjugate acid having a pKₐ of 11 or higher, or 11 to 18, or 11 to 16. The counterion may be a monoanion or a polyanion. For example, the counterion may be a monoanion, such as the hydroxide anion or an alkoxide anion. Example alkoxide anions include methoxide and ethoxide anions. Other example alkoxide anions include those containing 3, 4, 5, 6, 7, 8, 9 or 10 carbon atoms, either linearly or branched. For example, the alkoxide anions may comprise 1 to 4, or 1 to 6, or 1 to 8 or I to 10 carbon atoms.

The cationic methylated salts may optionally comprise a linker group between the sulfur, selenium, or phosphorous atom of the salt and the solid support. The linker group may comprise any groups capable of bonding to the sulfur, selenium or phosphorous atoms and immobilizing the salt as a whole to the solid support. In one embodiment, the linker group may comprise an alkyl group, an aryl group, a heteroaryl group, or any combination thereof either linearly or branched.

An “alkyl” group according to the invention is a straight-chain or branched-chain hydrocarbon radical having from 1 to 20 carbon atoms, for example 1 to 10 carbon atoms, for example 1, 2, 3, 4, 5, 6, 7, 8, 9 or 10 carbon atoms. For instance, the alkyl group may comprise 1 or 2, or 1 to 4, or 1 to 6, or 1 to 8, or 1 to 10, or 2 to 10, or 4 to 10, or 6 to 10 or 8 to 10 carbon atoms. An “alkyl” group also may be a cyclic group, for example 1 ring, or 2 or more fused rings. The rings can contain, for example, 5 or 6 carbon atoms in each ring. An “alkyl” group may be saturated, unsaturated or partially unsaturated. Moreover, any methylene (—CH₂—) group, or each of two or more non-adjacent methylene groups, in the alkyl group may be replaced by an oxygen atom.

An “aryl” group according to the invention is an aromatic carbocyclic ring or fused carbocyclic ring structure that comprises at least one benzene ring. The aryl group may contain, for example, 1 ring. The aryl group could also contain, for example, 2 or more fused rings, such as 2, 3 or 4 rings. When the aryl group contains multiple rings, each ring may contain, independently, 5 or 6 carbon atoms Examples of aryl groups include phenyl, naphthyl, 1, 2, 3, 4-tetrahydronaphthyl and indenyl groups.

A “heteroaryl” group according to the invention is an aromatic ring or fused ring structure wherein one or more carbon atoms of the ring structure are replaced by O, N, or S. The heteroaryl group may contain, for example, 1 ring. The heteroaryl group could also contain, for example, 2 or more fused rings, such as 2, 3, or 4 rings. When the heteroaryl group contains multiple rings, each ring may contain, independently, 5 or 6 ring atoms. Example heteroaryl groups include pyridinyl, thienyl, and isoquinolinyl groups.

The alkyl, aryl, and heteroaryl groups may be unsubstituted or substituted by one or more further alkyl, aryl or heteroaryl groups. Any alkyl, aryl or heteroaryl group may be unsubstituted or substituted by one or more further alkyl groups, one or more electron withdrawing groups, one or more electron donating groups, or combinations thereof. Furthermore, any fused ring alkyl, aryl or heteroaryl group may be substituted on all rings, or on some rings but not others, with the substituents being identical or different.

An “electron withdrawing group” is a substituent that draws electrons to itself more than a hydrogen atom would if it occupied the same position. Example electron withdrawing groups include halogen, nitro, cyano, hydroxyl, fluoroalkyl, perfluoroalkyl, nitrile, carboxyl, carboxylic ester, amide, sulfoxide, sulfone, carbonyl and ammonium groups. The carbonyl groups may exist as ketones or as aldehydes. “Halogen” according to the invention means fluorine, chlorine, bromine, or iodine. The “alkyl” in “fluoroalkyl” and “perfluoroalkyl” according to the invention takes the meaning of “alkyl” as defined above. An “electron donating group” is a substituent that draws electrons to itself less than a hydrogen atom would if it occupied the same position. Example electron donating groups include alkoxy groups, for example, methoxy and ethoxy groups. The “alk” in “alkoxy” according to the invention takes the meaning of “alkyl” as defined above.

The resins need not comprise a linker group between the sulfur, selenium or phosphorous atom and the solid support. Instead, the cationic salt may consist only of the cationic sulfonium, sulfoxonium, selenonium or phosphonium group immobilized directly on the solid support, for example immobilized directly to the phenyl ring of a polystyrene-based resin. Resins in this instance include those that would result from the methylation of poly(4-thiomethyl)styrene or poly(3-thiomethyl)styrene. A resin derived from methylation of poly(2-thiomethyl)styrene may also be acceptable, but, depending on the overall structure of the resin components, may be less desirable due to potential steric hinderance of the approach to the sulfur atom caused by the polymeric backbone of the solid support.

The alkyl, aryl and heteroaryl groups and their substituents, whether those groups are present in the linker groups or are substituents on the sulfur, selenium or phosphorous atoms of the salts, can be chosen so as to preserve the utility of the resin, and to achieve desired reactivity properties of the resin; i.e., through “tuning” of the reactivity.

Whether present in a linker group or in a substituent on the sulfur, selenonium or phosphorous atoms of the resins, functional groups in the near vicinity of the sulfur, selenonium and phosphorous atoms can be chosen to avoid deprotonation of the carbon bearing the relevant atom, and to avoid deprotonation at a position β- to the atom that would given an elimination reaction of the sulfonium, sulfoxonium, selenonium or phosphonium group. Example functional groups that could potentially cause such deprotonation, depending on the overall structure of the resin components, include ester, ketone, nitrile and nitro groups.

When a linker group is present, the groups in the linker can be chosen so as to not incorporate groups that would chemically react with the solid support, with other groups on the cationic methylated salt itself, or with methylating agents that are used to generate the resin from its precursors. Groups that may potentially create such undesirable reactions may include, depending on the overall structure of the resin components, amines, carboxylates and phenols.

For substituents in a linker group that are placed on a ring in an ortho-position to the attachment of the sulfur, selenonium or phosphorous atom, those substituents can be chosen to be of such size and number that would not prevent complexation of the alkylation substrate, and that would not prevent the desired methylation. Thus, depending on the overall structure of the resin components, it may be less desirable to incorporate two alkyl groups in both ortho-positions relative to the relevant atom. It may also be less desirable to incorporate a sterically-hindering group ortho to the atom, such as a tertiary butyl group, depending on the overall structure of the resin components.

The substituents on alkyl groups that are either used in the linker group or as substituents on the sulfur, selenonium or phosphorous atom, can be chosen to avoid interfering with the target methylation reaction. Substituents that may cause such interference, depending on the overall structure of the resin components, include phenols, thiols and carboxylates. Likewise, any degree of branching in the alkyl groups may be chosen to avoid undue interference with the functioning of the resin, such as the desired binding of a phenolate to a sulfonium group of the resin. It may also be desirable under the appropriate circumstances to avoid double branching of groups attached to the carbon bearing the sulfur, selenonium or phosphorous atom, if a certain possibility exists of unimolecular decomposition of the resin. Furthermore, and depending on the overall structure of the resin components, it may be desirable to avoid using an alkyl group other than methyl as a substituent on the sulfur, selenonium or phosphorous atom, to avoid having such a group transferred to the target in preference to the methyl group.

The types of alkyl, aryl and heteroaryl groups and their substituents may be chosen to increase or decrease reactivity of the resin. An increase in reactivity may be desirable so as to decrease the temperature or time necessary for the methylation reaction. A decrease in reactivity may be desirable if there are compounds present that can also undergo the methylation reaction at a rate that is comparable to the target, such as morphine. For example, if carboxylic acids are bound to the resin and undergo the methylation reaction, they will form methyl esters that are released from the resin to contaminate the desired product.

To a certain extent, the reactivity of the resin may be “tuned” by choice of substituents on the sulfur, selenium or phosphorous atom. For example, RR′SCH₃⁺ will be less reactive than ArRSCH₃⁺, which will in turn be less reactive than ArAr′SCH₃⁺, where R and R′ are alkyl groups, Ar and Ar′ are aryl or heteroaryl groups, and R and Ar′ are substituents in addition to the indicated methyl group. This follows from the electron withdrawing effects (inductive and resonance) that aryl groups have on heteroatom basicity; increasing the number of aryl groups will decrease the basicity of the sulfur, thereby making it a better leaving group.

More subtle “tuning” of the reactivity of the resin may be accomplished by varying the nature of substituents present on the aryl or heteroaryl groups. For example, an electron withdrawing group para to the sulfur will increase its leaving group ability and thereby facilitate the methylation reaction. Electron withdrawing groups that could be used for this purpose include nitro, nitrile, carboxylic acid, carboxylic ester, amide, sulfoxide, sulfone, carbonyl and ammonium. Reactivity could be modified by placing the electron withdrawing groups in other places on the ring, and combinations of electron withdrawing and/or electron donating groups could be used to modify reactivity. Furthermore, if a diarylmethylsulfonium (ArAr′SCH₃⁺) were utilized, either or both of the Ar rings could incorporate reactivity modifying groups.

Another embodiment of the invention is a resin comprising at least one cationic methylated salt immobilized on a solid support as in formula (Ia), (Ib) or (Ic):

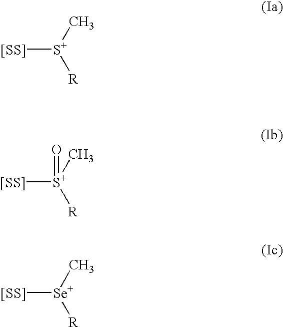

wherein

[SS] is the solid support; and

R is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof, and

wherein any methylene group, or each of two or more non-adjacent methylene groups, in an alkyl group in R may be replaced by an oxygen atom, and

wherein the indicated sulfur and selenium atoms are bound to carbon atoms.

A subgroup of the formulas (Ia), (Ib) and (Ic) includes R defined as —CH₃, and [SS] defined as a polymer.

Another embodiment of the invention is a resin comprising at least one cationic methylated salt immobilized on the solid support as in formula (IIa), (IIb) or (IIc):

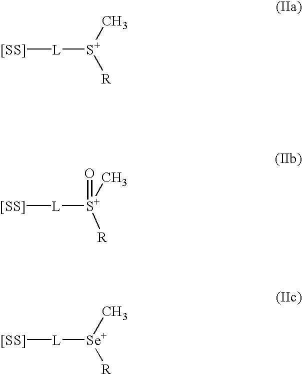

wherein

[SS] is the solid support and L is a linker group; and

L, R each independently of the other is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof; and

wherein any methylene group, or each of two or more non-adjacent methylene groups, in an alkyl group in L or R may be replaced by an oxygen atom, and

wherein the indicated sulfur and selenium atoms are bound to carbon atoms.

A subgroup of the formulas (IIa), (IIb) and (IIc) includes L and R each defined independently of the other as an alkyl group, which is unsubstituted or substituted as recited above. Alternatively, R may be defined as —CH₃ when L is defined as an aryl group, such as phenyl. [SS] also may be defined as, for example, a polymer.

Other subgroups of the formulas (IIa), (IIb) and (IIc) include the following:

L and R are each independently of the other an aryl or heteroaryl group, in which the aryl and heteroaryl groups are unsubstituted or substituted as recited in formulas (IIa), (IIb) and (IIc);

L is an alkyl group and R is an aryl or heteroaryl group, in which the alkyl, aryl and heteroaryl groups are unsubstituted or substituted as recited in formulas (IIa), (IIb) and (IIc);

L is an aryl or heteroaryl group and R is an alkyl group, in which the alkyl, aryl and heteroaryl groups are unsubstituted or substituted as recited in formulas (IIa), (IIb) and (IIc);

L, R, or both L and R are each independently of the other an aryl group that contains one ring, which is unsubstituted or substituted as recited in formulas (IIa), (IIb) and (IIc);

L, R, or both L and R are each independently of the other an aryl group that contains two or more fused rings, wherein each ring is unsubstituted or substituted as recited in formulas (IIa), (IIb) and (IIc);

L, R, or both L and R are each independently of the other phenyl or naphthalene, which is unsubstituted or substituted as recited in formulas (IIa), (IIb) and (IIc);

L, R, or both L and R are each independently of the other a heteroaryl group that contains one ring, which is unsubstituted or substituted as recited in formulas (IIa), (IIb) and (IIc);

L, R, or both L and R are each independently of the other a heteroaryl group that contains two or more fused rings, wherein each ring is unsubstituted or substituted as recited in formulas (IIa), (IIb) and (IIc); and

L, R, or both L and R are each independently of the other pyridine or quinoline, which is unsubstituted or substituted as recited in formulas (IIa), (IIb) and (IIc).

Another embodiment of the invention is a resin comprising at least one cationic methylated salt immobilized on the solid support as in formula (IId):

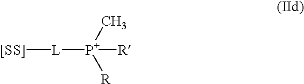

wherein

[SS] is the solid support and L is a linker group; and

L, R, R′ each independently of the other is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof; and

wherein any methylene group, or each of two or more non-adjacent methylene groups, in an alkyl group in L, R or R′ may be replaced by an oxygen atom, and

wherein the indicated phosphorous atom is bound to carbon atoms.

A subgroup of formula II(d) includes R and R′ both defined as —CH₃. In another subgroup, at least one or R and R′ is defined as an aryl or heteroaryl group, which may be unsubstituted or substituted as recited in formula II(d). In another subgroup, L is defined as an aryl or heteroaryl group, which may be unsubstituted or substituted as recited in formula II(d).

Another embodiment of the invention is a resin comprising at least one cationic methylated salt immobilized on the solid support as in formula (IIa) or (IIIb):

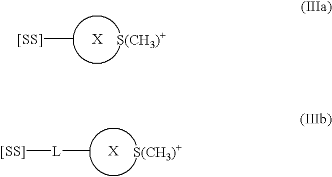

wherein

[SS] is the solid support and L is a linker group;

L is an alkyl, aryl or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof, and

X is a 5 to 10 membered aromatic or non-aromatic ring that may contain, in addition to the —S(CH₃)⁺ group, one or more atoms chosen from S, N and O,

which is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof,

wherein any methylene group, or each of two or more non-adjacent methylene groups, in an alkyl group in L or X may be replaced by an oxygen atom, and

wherein the indicated sulfur atom is bound to carbon atoms.

A subgroup of the formulas (IIIa) and (IIIb) includes [SS] defined as a polymer.

Example rings X in formulas (IIIa) and (IIIb) include thiophene, thiazole, dibenzothiophene and thianaphthene.

Another embodiment of the invention is a resin comprising at least one cationic methylated salt immobilized on the solid support as in formula (IVa) or (IVb):

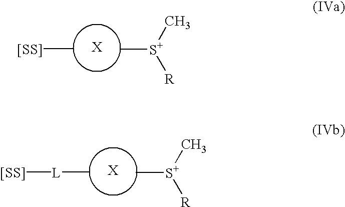

wherein

[SS] is the solid support and L is a linker group;

L, R each independently of the other is an alkyl, aryl or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof, and

X is a 5 to 10 membered non-aromatic carbocyclic or heterocyclic group,

which is unsubstituted or substituted by one or more further alkyl, aryl or heteroaryl groups, and

in which any alkyl, aryl and heteroaryl group may be unsubstituted or substituted by one or more further alkyl groups, one or more electron withdrawing groups, one or more electron donating groups, or a combination thereof,

wherein any methylene group, or each of two or more non-adjacent methylene groups, in an alkyl group in L, R or X may be replaced by an oxygen atom, and

wherein the indicated sulfur atom is bound to carbon atoms.

A subgroup of the formulas (IVa) and (IVb) includes [SS] defined as a polymer.

Example rings X in formulas (IVa) and (IVb) include cyclopentane and cyclohexane, with those groups being unsubstituted or substituted as recited in formulas (IVa) and (IVb).

Another embodiment of the invention is a resin comprising

a solid support, and

at least one cationic methylated sulfonium, sulfoxonium or selenonium salt immobilized on the solid support,

in which the counterion of the at least one cationic methylated salt is an anion that has a conjugate acid having a pKₐ of 10 or higher,

wherein the at least one cationic methylated salt is immobilized on the solid support as in formula (Va), (Vb) or (Vc):

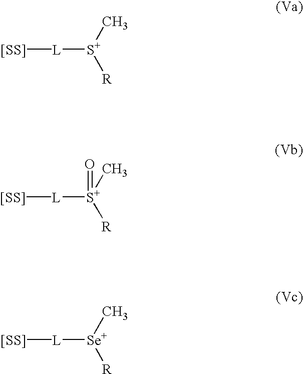

wherein

[SS] is the solid support and L is a linker group; and

L, R each independently of the other is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,

wherein any methylene group, or each of two or more non-adjacent methylene groups, in an alkyl group in L or R may be replaced by an oxygen atom, and

wherein the indicated sulfur and selenium atoms are bound to carbon atoms.

In a subgroup of the formulas (Va), (Vb), (Vc) and (Vd), L is unsubstituted or substituted phenyl and R is —CH₃. In another subgroup, [SS] a polymer, such as polystyrene. In another subgroup, the counterion to the at least one cationic methylated salt is the hydroxide ion, or an alkoxide ion such as a methoxide or ethoxide ion.

Another embodiment of the invention is a resin comprising

a solid support, and

at least one cationic methylated sulfonium salt immobilized on the solid support,

in which the counterion of the at least one cationic methylated salt is an anion that has a conjugate acid having a pKₐ of 10 or higher,

wherein the at least one cationic methylated salt is immobilized on the solid support as in formula (VI):

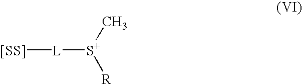

wherein

[SS] is the solid support and L is a linker group; and

L, R each independently of the other is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,

wherein any methylene group, or each of two or more non-adjacent methylene groups, in an alkyl group in L or R may be replaced by an oxygen atom, and

wherein the indicated sulfur atom is bound to carbon atoms.

In a subgroup of formula (VI), L is unsubstituted or substituted phenyl and R is —CH₃. In another subgroup, [SS] is a polymer, such as polystyrene. In another subgroup, the counterion to the at least one cationic methylated salt is the hydroxide ion, or an alkoxide ion such as a methoxide or ethoxide ion.

The resins of the invention may be prepared, for example, by forming a resin precursor of the formula [SS]-L-YCH₃, methylating the precursor to form [SS]-L-Y(CH₃)₂⁺, and, if necessary, converting that product to a form in which the counterion of the at least one cationic methylated salt is an anion that has a conjugate acid having a PKₐ of 10 or higher. For example, the final resin may be in its hydroxide or alkoxide form. In the general formulas above, [SS] is a solid support, L is a linker group or does not exist, and Y is S, ΔS═O, or Se. The preparation of cationic methylated phosphonium resins proceeds as above, differing in the preparation of, for example, a precursor of the formula [SS]-L-P(CH₃)₂ and methylating the precursor to form [SS]-L-P(CH₃)₃⁺. Furthermore, the initial —CH₃ group or groups in the precursors [SS]-L-YCH₃ and [SS]-L-P(CH₃)₂ could just as well be replaced by, for example, other unsubstituted or substituted alkyl, aryl or heteroaryl groups, without meaningful change in the preparation procedures.

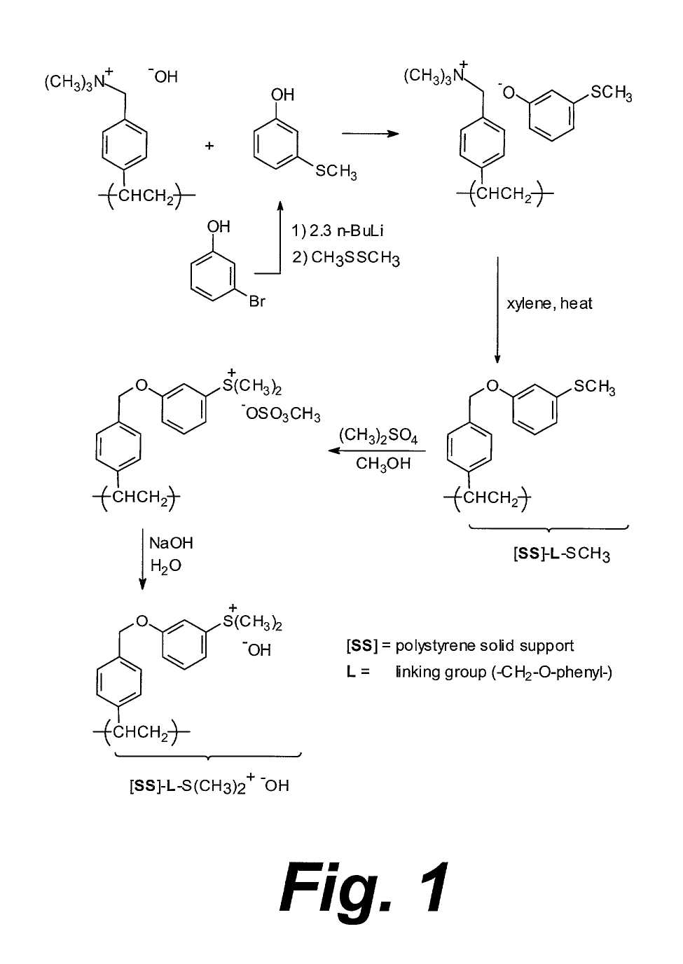

FIG. 1 illustrates one example synthesis that follows the general procedure discussed above. In this embodiment, an aryl thioether ArSCH₃ is covalently attached to a solid support to give [SS]-L-SCH₃, where [SS] is a polystyrene solid support and L is the linker group —CH₂—O-phenyl-. This is accomplished by reaction of an alcoholic (for example, methanolic) solution of 3-methylthiophenol (formed by successive treatment of 3-bromophenol with greater than 2 molar equivalents n-butyl lithium and dimethyl disulfide) with the hydroxide form of a macroreticular strong anion exchange resin possessing benzyltrimethylammonium groups (for example, the hydroxide form of Amberlyst A-26). Replacement of the alcohol solvent with a high boiling nonpolar organic solvent (for example, xylene) is followed by heating to reflux (for example, for 10-15 hours), to drive off the remaining traces of the alcohol solvent, and to effect displacement of the trimethylamine group of the resin by the phenoxide oxygen. The resulting product is the resin precursor [SS]-L-SCH₃.

A resin as above could also be prepared from reaction of commercially available poly(4-chloromethyl)styrene with 3-methylthiophenol/base. Furthermore, and although the synthesis of 3-methylthiophenol above is quite rapid and convenient, the large scale preparation of this compound could also be carried out by diazotization of 3-aminophenol, reaction of the diazonium salt with ethyl xanthate, and hydrolysis to give 3-mercaptophenol, which will be methylated on sulfur with a single molar equivalent of an appropriate methylating agent, such as dimethyl sulfate, bromomethane, or iodomethane.

The toluene or other nonpolar solvent can then be replaced by methanol, and the sulfur of the resin precursor can be methylated using dimethylsulfate. The resulting methylated resin [SS]-L-S(CH₃)₂⁺ CH₃OSO₃⁻ can be converted to its hydroxide form [SS]-L-S(CH₃)₂^{+ —}OH by passage of aqueous lithium, sodium or potassium hydroxide through the resin, or to its methoxide form [SS]-L-S(CH₃)₂+-OCH₃ by passage of methanolic lithium, sodium or potassium methoxide through the resin. This alkoxide or methoxide form of the methylated resin constitutes one example of a resin of the invention.

A alternative synthesis to that in FIG. 1 can involve addition of methanethioi (or other alkanethiol) to the double bond of the product eugenol, promoted by any of a wide variety of free radical initiators (for example, azoisobutyronitrile, AIBN, used in toluene or other solvent at reflux) to give the thioalkyl derivative. This can be loaded as a methanol solution onto a benzyltrimethylammonium anion exchange resin in the hydroxide form and heated as above (for example, subsequent to a solvent change to toluene) to form a methyisulfonium methylation resin precursor of the form [SS]-L-SCH₃. Methylation and conversion to the hydroxide salt will give a methylsulfonium methylation resin, [SS]-L-S(CH₃)₂^{+ -}OCH₃. In an alternative embodiment of this attachment method, benzenethiol, or another arylthiol can be added to the double bond of eugenol. Attachment to the exchange resin as described above will give a methylsulfonium methylation resin precursor of the form [SS]-L-ArS. Subsequent methylation and conversion to the methoxide salt will give a Methylsulfonium Methylation Resin [SS]-L-ArSCH₃^{+ -}OCH₃ that is a more active methylating agent than the [SS]-L-S(CH₃)₂^{+ -}OCH₃ described previously.

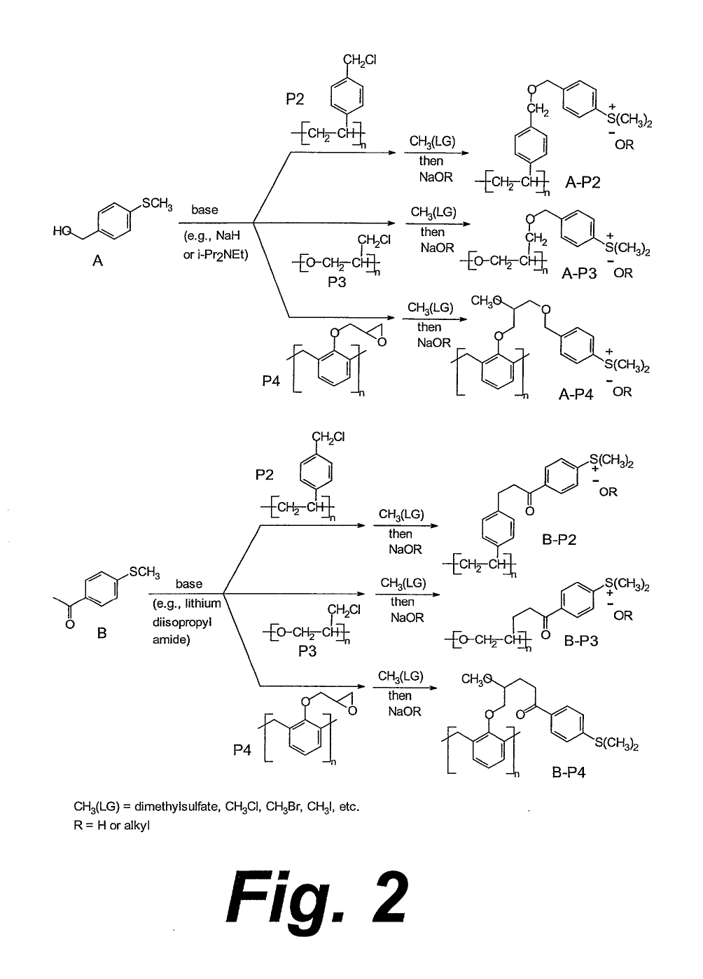

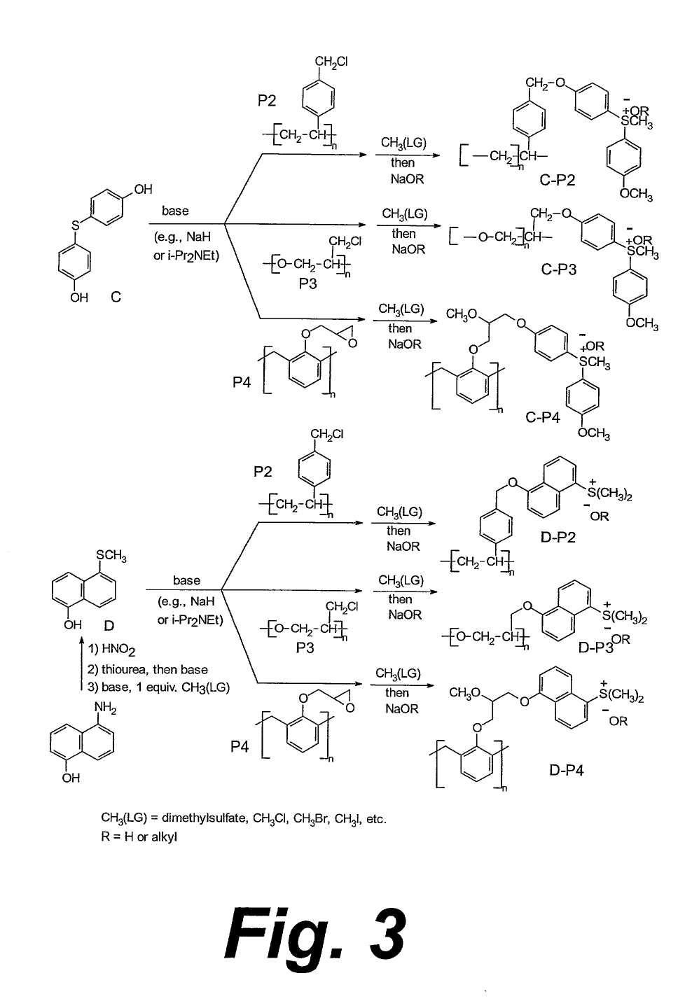

FIGS. 2 and 3 illustrate ways to prepare other methylsulfonium resins of the invention from commercially available starting materials. Each case involves the reaction of a nucleophile on a methythio compound with an electrophile on a polymer. The Figures illustrate the use of commercially available polymers poly(4-chloromethyl)styrene (“P2”, also known as Merrifeld's resin), polyepichlorohydrin (“P3”) and poly(phenylglycidyl ether)-co-formaldehyde (“P4”).

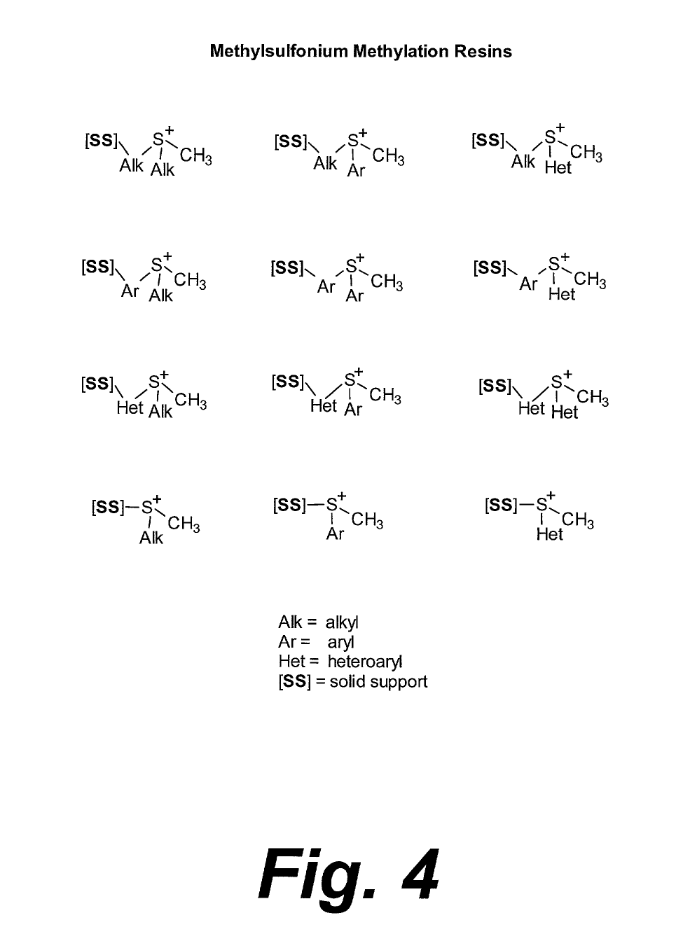

The sulfonium portions of the in FIGS. 2 and 3 also illustrate a number of different types of linker groups. Polymers derived from “A” have alkyl substitution on the phenyl ring attached to the methylsulfonium. Polymers derived from “B” have an electron-withdrawing group para to the sulfonium thereby increasing its reactivity. Polymers derived from “C” have both additional phenyl substitution (which increase reactivity of the sulfonium as a methylating agent) and electron donating groups that assist in making the methylation reagent, and serve as a convenient point of attachment. Polymers derived from “D” have a fused aromatic ring. A wide variety of methylated sulfonium resins, such as those illustrated in FIG. 4, may be prepared in similar fashion to those synthesized in FIGS. 1-3.

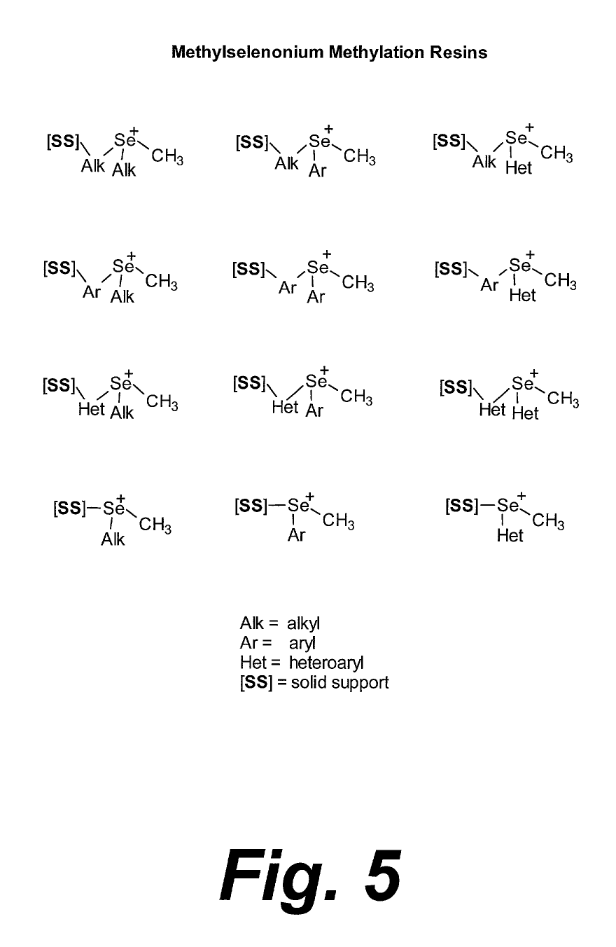

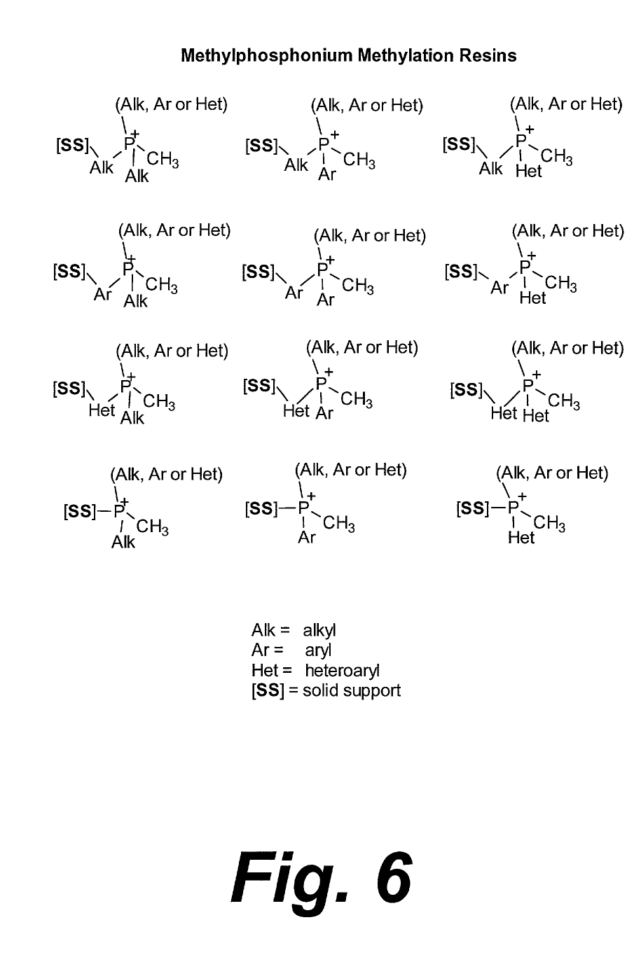

Although the embodiments described above utilize a methyl sulfonium group as the methylating agent, methyl selenonium salts could also be employed to give methylselenonium methylation resins such as those illustrated in FIG. 5 when localized on the appropriate solid support. Indeed, based on the position of selenium on the period table, these compounds should be more readily formed from their non-methylated precursors than the sulfur analogs, and they should be more reactive as methylating agents than their sulfonium analogs. Similarly, phosphonium salts attached to solid supports, such as those illustrated in FIG. 6, could constitute methylphosphonium methylating resins that could be superior in their methyl transferring abilities compared to the nitrogen analogs, while being more easily prepared from the non-methylated precursors.

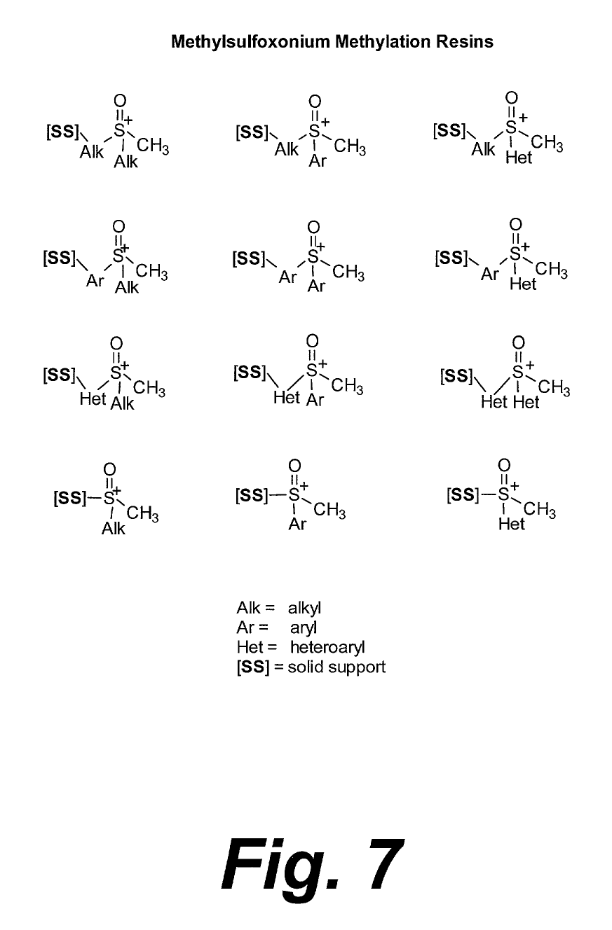

Another class of alkylating agents are those resins incorporating methylsulfoxonium groups, including those resins illustrated in FIG. 7. For all resins illustrated in FIGS. 4-7, the substituents Alk, Ar and Het may be unsubstituted or substituted alkyl, aryl or heteroaryl groups. The methylated selenonium, phosphonium, and sulfoxonium resins discussed above could be prepared analogously to the preparation of methylsulfonium reins. For example, the methylated selenonium, phosphonium, and sulfoxonium resins may be prepared from the corresponding precursors lacking a methyl group through methylation reactions using dimethylsulfate or other methylating agent. The corresponding methylselenonium, methylphosphonium and methylsulfoxonium methylation resin precursors, in turn, could be formed, for example, in fashions analogous to the formation of the methylsulfonium methylation resin precursor.

The discussion above details the use of dimethylsulfate as the methylating agent for formation of the methylated sulfonium, sulfoxonium, selenonium and phosphonium salts with the ⁻OSO₃CH₃ counterion. Any of a wide variety of other methylating agents could, alternatively, be employed for this purpose, including iodomethane, bromomethane, chloromethane or any of a number of methyl sulfonate esters, such as methyl sulfonate or methyl p-toluenesulfonate. In these instances, similar methylation resins would result, with different counterions (for example, I⁻ when CH₃l was used); and these could be converted to the corresponding hydroxide or methoxide form, for example, as described above.

The discussion above also mentions conversion of the initially formed methylation resins having the counterion ⁻OSO₃CH₃ to the corresponding methoxide or hydroxide forms. This has the advantage that simple passage of a solution of a neutral phenol, such as morphine, through the resin will result in deprotonation of the phenol with concomitant binding of the phenolate to the resin. In an alternative embodiment, a solution of the phenol (or carboxylic acid or thiol, depending on the target for methylation) that has been adjusted to a pH value such that the target for alkylation exists as its phenolate (or carboxylate, or thiolate) is applied to the methylation resin that is still in its ionic form resulting from the initial methylation reaction, that is [SS]-RR′YCH₃^{+ -}OSO₃CH₃, [SS]-ArRYCH₃^{+ -}OSO₃CH₃ or [SS]-ArAr′YCH₃^{+ -}OSO₃CH₃ (Y═S, Se, PR or PAr) when dimethylsulfate was the original methylating species. Simple ion exchange will result in the formation of the desired ion pair [SS]-RR′YCH₃^{+ -}O-Substrate, [SS]-ArRYCH₃^{+ -}O-Substrate or [SS]-ArAr′YCH₃^{+ -}O—Substrate (Y═S, Se, PR or PAr), in which —O-Substrate is the anion of the target for methylation (e.g., for targets such as phenols, carboxylic acids; for thiols, one will have ⁻S-substrate).

The resins of the invention may be used as methylation resins. Thus, another embodiment of the invention is a process for methylating the phenolic moiety of a compound, which comprises:

loading the compound onto a resin of the invention, and

heating the loaded resin in a solvent under conditions sufficient to methylate the phenolic moiety of the compound.

This method applies, for example, to methylating the phenolic moiety of morphine to form codeine.

The use of the resins of the invention in the methylation process described above can carry significant advantages, from a chemical reactivity standpoint, compared to nitrogen-based resins. Without wishing to be bound to any particular scientific theory, the following discussion attempts to explain some possible sources of those advantages. The methylation reaction involved in the morphine to codeine conversion, for instance, is an example of a nucleophilic substitution reaction, specifically an S_{N}2 reaction. For a given alkyl group and nucleophile (methyl and phenolate, respectively, in the morphine to codeine conversion), the reaction is dependent on the leaving group, the solvent and the temperature. If all other things are substantially equal, basicity generally decreases as one moves down a column in the periodic table. Thus, H₂O is more basic than H₂S, which is more basic than H₂Se. Similarly, NH₃ is more basic than PH₃. Increasing leaving group ability can be correlated with decreasing basicity, and thus one has increasingly good leaving groups in the series H₂O<H₂S<H₂Se, and NH₃<PH₃. For example, ArP(CH₃)₂ could be a much superior leaving group to ArN(CH₃)₂, where Ar is an aryl or heteroaryl group. One also has increasingly good leaving groups for compounds in which alkyl is replaced by aryl groups. For example, ArArS(CH₃) could be a much superior leaving group to ArS(CH₃)₂, where Ar is an aryl or heteroaryl group.

In addition to leaving group ability, and if all other things are substantially equal, nucleophilicity can increase as one goes down a column in the periodic table. This could possibly be explained as consequence of increasing polarizability, and can lead to the nucleophilic ordering R₂Se>R₂S>R₂O. The combination of leaving group ability and nucleophilicity leads to the counterintuitive conclusion that in the series R₂O, R₂S, R₂Se one has both increasing nucleophilicity and increasing leaving group ability. The consequence of this conclusion can have significant implications for the conversion of phenolic compounds such as morphine to their methyl ethers.

The methylsulfonium procedure of the invention, for example, shows some improved characteristics over the original '750 patent procedure in at least the step [SS]-L-S(CH₃)₂^{+ -}Morph→[SS]-L-SCH₃+ Codeine, where [SS] is a solid support, L is a linker group, and MorphO⁻ is the phenolic anion of morphine. Comparably substituted sulfur compounds are typically <10¹² as basic as the corresponding nitrogen compounds, making them vastly superior leaving groups. For example, the ′750 patent methylation procedure teaches heating the loaded resin at 105° for a period of nine hours in the strongly reaction promoting solvent toluene. In contrast, the methylation reaction of S(CH₃)₃^{+ -}OAr (with phenolate as ArO⁻) at 100° in the strongly reaction retarding solvent ethanol is reported to proceed at a rate of 48 M⁻¹hour⁻¹, corresponding to essentially complete reaction in 5-10 minutes (cf. Gleave, J. L.; Hughes, E. D.; Ingold, C. K., Journal of the Chemical Society, 1935, pp. 236-244). Use of ArS(CH₃)₃⁺ as the methylating agent should further accelerate the reaction due to the effect of the aryl group, allowing the methylation to proceed rapidly at a lower temperature. It should be quite practical to perform the methylation of codeine using the methylsulfonium procedure without changing the methanol solvent used in the loading of the opium extract on the column. Furthermore, this methylation can occur over a short period of time at the reflux temperature of methanol (65° C.), decreasing energy costs relative to those required for toluene, and increasing worker safety.

The benefits in the methylation step discussed above are not offset by any disadvantages in the preparation of the new methylsulfonium methylation resin. In fact, the methylation of a methylsulfonium resin precursor to the methylsulfonium methylation resin can proceed similarly to the corresponding step in the making of the nitrogen-based resins in the ′750 patent. This is because the nucleophilicities of ArSH and ArNH₂ are essentially identical, as are those of S(CH₃)₂ and NH₃. On this basis, the nucleophilicity of ArSCH₃ can be comparable to ArN(CH₃)₂.

One embodiment of the process using resins of the invention may comprise loading the resin in presence of a solvent, and heating the loaded resin in the presence of a solvent that is the same as that used during the loading. Another aspect of the process comprises heating the loaded resin in the presence of a solvent different from that used during the loading. In another embodiment of the process of the invention, the process employs the cationic methylated salts for the purpose of ion pairing with phenolic anions. This ion pairing capability serves to separate and thereby purify the methylation target from impurities that may be present.

In other embodiments of the process, the compound, for example morphine, is loaded onto the resin in the presence of a solvent that comprises water, or that comprises at least one alkanol, or that comprises both water and at least one alkanol. The “alk” in “alkanol” takes the same meaning as “alkyl” defined previously. Example alkanols include C₁-C₄ alkanols, for instance methanol and ethanol. The compound could also be loaded on the resin in the presence of a solvent that comprises at least one ketonic solvent, such as acetone or butanone. The compounds could also be loaded on the resin in the presence of a solvent that comprises tetrahydrofuran. In yet other embodiments of the process, the loaded resin is heated in the presence of a solvent that comprises at least one alkanol, such as methanol and ethanol. The loaded resin could also be heated in the presence of a solvent that comprises an aromatic hydrocarbon, such as toluene or benzene. The loaded resin could also be heated in the presence of a solvent that comprises a non-aromatic hydrocarbon and having a boiling point of ≦100° C., such as cyclohexane.

Still other embodiments of the process include loading the compound onto the resin in the presence of a solvent that comprises at least one alkanol, and heating the loaded resin in the present of a solvent that comprises at least one alkanol. This process can involve, for example, loading the compound onto the resin in the presence of a solvent that comprises methanol, ethanol, or both methanol and ethanol, and heating the loaded resin in the presence of a solvent that comprises methanol, ethanol, or both methanol and ethanol. Alternatively, the process can involve loading the compound onto the resin in the presence of a solvent that comprises methanol, ethanol, or both methanol and ethanol, and heating the loaded resin in the presence of a solvent that comprises toluene.

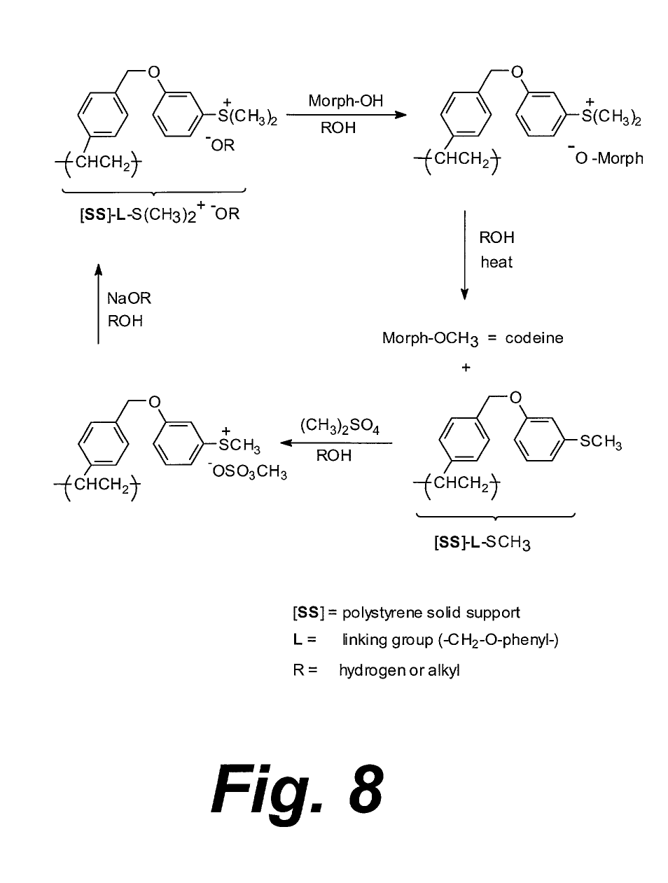

FIG. 8 illustrates an example process of the invention. As shown in FIG. 8, a solution of morphine (with Morph-OH representing the morphine molecule, which contains a phenolic —OH group) in a suitable solvent can be applied to, for example, a methylsulfonium methylation resin. The molecule undergoes an acid-base reaction with the basic counterion RO⁻ of the resin, and a majority of the phenolate derivative of morphine becomes bound as an ion pair to the resin in the form [SS]-L-S(CH₃)₂^{+ -}OMorph. Such ion pairing serves to separate and thereby purify the methylation target from impurities that may be present. Suitable solvents for use in this step include water, low molecular weight alcohols (for example, those with one to four carbons), low molecular weight ketonic solvents (for example, acetone, butanone), tetrahydrofuran, or other solvent capable of dissolving the morphine at a satisfactory concentration, but that is unlikely to react with the resin itself, or with common alkylating agents. One example solvent is methanol. The resin can then be washed with additional solvent, such as methanol, to remove species that are not ion paired with the methylsulfonium groups.

The loaded resin can then be heated to under conditions sufficient (for example 60-65° C. for a period of one to fifteen hours) to accomplish the nucleophilic substitution reaction (methylation reaction) to give codeine and methylsulfonium methylation resin precursor. The solvent can be drained and the methylsulfonium methylation resin precursor can be washed with methanol to remove remaining traces of codeine. Evaporation of the combined solvents obtained after the heating process will afford codeine.

Another embodiment of the invention comprises regenerating the expended resin for re-use after the methylation reaction. This embodiment comprises methylating the expended resin and converting it to its hydroxide or alkoxide form in the presence of water, methanol, ethanol, or combinations thereof. Thus, as shown in FIG. 8, the methylsulfonium methylation resin can be regenerated by realkylation of the methylsulfonium methylation resin precursor with dimethylsulfate, and then conversion to the methoxide or hydroxide form.

The use of a single solvent comprising methanol in the loading of morphine on the resin, the heating of the loaded resin, and the regeneration of the expended resin, carries several advantages. Methanol serves well for the morphine purification that leads up to the morphine to codeine conversion. It is also well suited for use in the regeneration step using dimethylsulfate. In contrast, utilization of non-polar solvents, such as toluene, can result in resin shrinkage and inaccessibility of the sulfide groups to alkylation by the methylating agent. Methanol is typically less expensive, and more easily disposable, than toluene. As discussed earlier, the use of a single solvent is also desirable from the standpoint of simplicity in manufacturing plant design, inventory requirements, environmental safety requirements. Other solvents may also be used in all steps of the process, including ethanol, which may be used, for example, in place of methanol or together with methanol in any or all of the process steps.

In addition to methanol and ethanol, other solvents might also be employed with equally good, or better results. Such solvents may, for example, provide favorable solubilities of morphine, codeine and their salts. Furthermore, while the use of a single solvent can be advantageous due to the simplicity, low cost, and enhanced safety of the resulting procedure, it may be the case that multiple solvents could be employed to better advantage, for example due to desirable solubility characteristics of the reactants or products. Regardless of the choice of solvent, the methylation resins of the invention should show markedly superior properties to nitrogen-based resins, such as higher reactivity in methylation reactions, and ease with which their precursors can themselves be methylated.

Another embodiment of the invention is a process for methylating the phenolic moiety of morphine to form codeine, which comprises:

a) loading morphine onto a resin which comprises

a solid support, and

at least one cationic methylated sulfonium salt immobilized on the solid support,

in which the counterion of the at least one cationic methylated salt is an anion that has a conjugate acid having a pKₐ of 11 or higher,

wherein the at least one cationic methylated salt is immobilized on the solid support as in formula (VI):

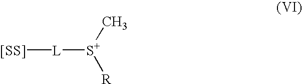

wherein

[SS] is the solid support and L is a linker group; and

L, R each independently of the other is an alkyl, aryl, or heteroaryl group,

in which the alkyl, aryl and heteroaryl group is unsubstituted or substituted by one or more further alkyl, aryl or heteroaryl groups, and

in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,

wherein any methylene group, or each of two or more non-adjacent methylene groups, in an alkyl group in L or R may be replaced by an oxygen atom, and

wherein the indicated sulfur atom is bound to carbon atoms,

in the presence of a solvent that comprises water, one or more alkanols, or water and one or more alkanols, and

b) heating the loaded resin in a solvent that comprises one or more alkanols under conditions sufficient to methylate the phenolic moiety of the compound to form codeine.

In one aspect of the process immediately above, the morphine can be loaded onto the resin in the presence of a solvent that comprises water, methanol, ethanol, or a combination thereof, and the loaded resin can be heated in a solvent that comprises methanol, ethanol, or a combination thereof. The heating step may occur, for example, at a temperature of 60-65° C., and for a period of one to fifteen hours, for example ten to fifteen hours.

The method discussed above may also be used for other methylation reactions on other species that are capable of existing as anions. The ability of the compounds to exist as anions ensures that the target group for methylation will be retained on the methylation resin through an ion pair interaction. Example species that fulfill this criteria include carboxylic acids and thiols. Application of carboxylic acids to a methylation resin, following by heating, will lead to the formation of the corresponding methyl esters. Application of thiols to the methylation resin, followed by heating, will lead to the formation of alkyl or aryl methylthioethers.

Thus, another embodiment of the invention is a process for converting a carboxylic acid moiety of a compound to its methyl ester, which comprises:

loading the compound onto a resin comprising:

a solid support, and

at least one cationic methylated sulfonium, sulfoxonium, selenonium or phosphonium salt immobilized on the solid support,

in which the counterion of the at least one cationic methylated salt is an anion that has a conjugate acid having a pKₐ of 7 or higher, and

heating the loaded resin in a solvent under conditions sufficient to form a methyl ester of the carboxylic acid moiety.

Yet another embodiment of the invention is a process for methylating a thiol compound, which comprises:

loading the compound onto a resin of the invention, and heating the loaded resin in the solvent under conditions sufficient to methylate the thiol compound.

Another embodiment of the invention is a resin that comprises:

a solid support, and

at least one cationic alkylated sulfonium, sulfoxonium, selenonium or phosphonium salt of 2 to 8 carbon atoms immobilized on the solid support,

in which the counterion of the at least one cationic alkylated salt is an anion that has a conjugate acid having a pKₐ of 10 or higher.

In one embodiment of this invention, the resin comprises an ethyl, propyl, butyl, isopropyl or isobutyl cationic salt. This resin may be used, for example, in the alkylation of phenols, carboxylic acids, or thiols with an alkyl group of 2 to 8 carbons atoms, for example 2, 3, 4, 5, 6, 7 or 8 carbon atoms. In another embodiment of this invention, the resin is used for the transfer of the alkyl groups being primary and unbranched in the β-position.

Another embodiment of the invention is a resin that comprises:

a solid support, and

at least one cationic benzylated sulfonium, sulfoxonium, selenonium or phosphonium salt immobilized on the solid support,

in which the counterion of the at least one cationic benzylated salt is an anion that has a conjugate acid having a pKₐ of 10 or higher.

In one embodiment of this invention, the resin comprises an unsubstituted or substituted benzylated cationic salt. This resin may be used, for example, in the benzylation of phenols, carboxylic acids, or thiols. This alternative embodiment could be accomplished, for example, by reacting [SS]-RR′Y, [SS]-ArRY or [SS]-ArAr′Y (Y═S, Se, PR or PAr) with an unsubstituted or substituted benzyl halide ArCH₂X (X=halide, sulfonate ester or other leaving group), where R and R′ are unsubstituted or substituted alkyl groups and Ar is an unsubstituted or substituted aryl or heteroaryl group.

Another embodiment of the invention is a resin that comprises:

a solid support, and

at least one cationic allylated sulfonium, sulfoxonium, selenonium or phosphonium salt immobilized on the solid support,

in which the counterion of the at least one cationic allylated salt is an anion that has a conjugate acid having a pKₐ of 10 or higher.

This resin may be used, for example, in the allylation of phenols, carboxylic acids, or thiols.

The alkylated, benzylated, and allylated resins of the invention can be made in an analogous manner to the methylated resins of the invention, with the appropriate modifications in the syntheses to alkylate, benzylate, or allylate the sulfur, selenonium or phosphonium groups of the salts. Furthermore, the alkylation, benzylation and allylation processes may be performed in like manner to the methylation reactions described herein.

### EXAMPLE 1

### Preparation of 3-methylthiophenol

The preparation of 3-methylthiophenol was conducted under standard conditions for air and moisture sensitive reactions (oven dried flasks, nitrogen atmosphere, dry solvents). n-Butyllithium (13 mL of a 2 M solution in hexanes) was added to a solution of 3-bromophenol (2.0 g) in ether (50 mL) at such a rate as to avoid reflux of the ether. After stirring the resulting solution for four hours at room temperature, dimethyl disulfide was added, and the resultant mixture was stirred an additional two hours. After cautious addition of water (˜25 mL), concentrated hydrochloric acid was added to give an aqueous acidity of pH 3. Separation of the aqueous and organic layers was followed by drying of the organics with sodium sulfate. Removal of the ether by rotary evaporation gave substantially pure 3-methylthiophenol (1.8 g).

### EXAMPLE 2

### Preparation of the Methylsulfonium Methylation Resin

Amberlite A-26 in the hydroxide form (2.0 g dry weight) and a solution of 3-methylthiophenol in 70 mL dry methanol were shaken in a sealed vessel for approximately 12 hours. The resulting resin was filtered and then washed thoroughly with methanol (˜125 mL). The resin was then covered with xylene (100 mL) and heated at reflux for twenty-eight hours. The cooled mixture was filtered, and the resin washed thoroughly with methanol (˜125 mL). Methanol (100 mL) and dimethyl sulfate (10 mL) was added to the resin, the vessel sealed, and the mixture shaken twenty-four hours. After cautiously releasing pressure from the vessel, the resin was filtered and washed successively with aqueous sodium chloride (1 M, 50 mL), water (50 mL) and aqueous sodium hydroxide (1 M, 50 mL). The resin was covered with sodium hydroxide (1 M) for three hours, then filtered, rinsed with water to neutrality, and rinsed again with methanol to give the methylsulfonium methylation resin in its hydroxide/methoxide form.

### EXAMPLE 3

### Methylation Resins

The following resins, for example, may be prepared following the syntheses described throughout this specification:

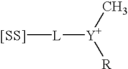

| Resin | [SS] | L | Y | R |
|---|---|---|---|---|
| A | Polymer | 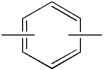 | —S— | —CH₃ |
| B | Polymer | 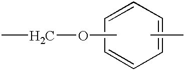 | —S— | —CH₃ |
| C | Polymer | 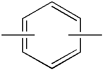 | —S— | 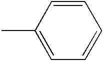 |
| D | Polymer | 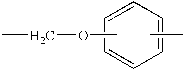 | —S— | 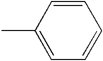 |
| E | Polymer | —(CH₂)—ₙ, n = 1 to 5 | —S— | 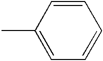 |
| F | Polymer | 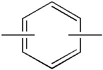 | —S(O)— | —CH₃ |
| G | Polymer | 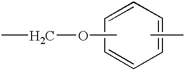 | —S(O)— | —CH₃ |
| H | Polymer | 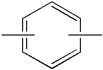 | —S(O)— | 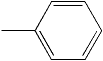 |
| I | Polymer | 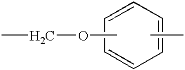 | —S(O)— | 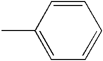 |
| J | Polymer | —(CH₂)—ₙ, n = 1 to 5 | —S(O)— | 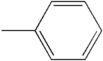 |
| K | Polymer | 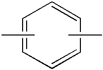 | —Se— | —CH₃ |
| L | Polymer | 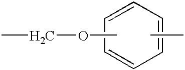 | —Se— | —CH₃ |
| M | Polymer | 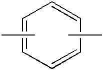 | —Se— | 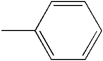 |
| N | Polymer | 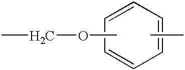 | —Se— | 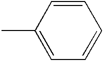 |
| O | Polymer | —(CH₂)—ₙ, n = 1 to 5 | —Se— | 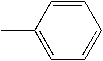 |
| P | Polymer | 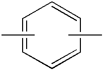 | —P(CH₃)—or —P(phenyl)- | —CH₃ |
| Q | Polymer | 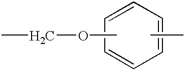 | —P(CH₃)—or —P(phenyl)- | —CH₃ |
| R | Polymer | 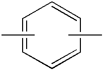 | —P(CH₃)—or —P(phenyl)- | 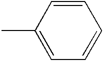 |
| S | Polymer | 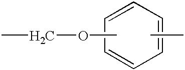 | —P(CH₃)—or —P(phenyl)- | 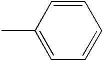 |
| T | Polymer | —(CH₂)—ₙ, n = 1 to 5 | —P(CH₃)—or —P(phenyl)- | 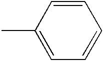 |

### EXAMPLE 4

### Conversion of Morphine to Codeine

The hydroxide form of the methylsulfonium methylation resin was covered with methanol containing morphine (0.21 g) for approximately sixty hours. Filtration of the resin was followed by a methanol wash. Evaporation of these combined filtrates demonstrated complete adsorption of the morphine by the resin. The morphine-loaded resin was combined with toluene (125 mL) and heated until all of the methanol had distilled out. Heating was continued for 1.5 hours, when analysis of the supernatent liquid by high performance liquid chromatography showed a single peak corresponding to codeine. After cooling, the resin was filtered, washed with toluene (˜25 mL), and the combined filtrates evaporated to give codeine as a white solid (0.200 g, 91%).

We claim:

1. A resin, comprising:
a solid support, and
at least one cationic methylated sulfonium, sulfoxonium or selenonium salt immobilized on the solid support,
in which the counterion of the at least one cationic methylated salt is an anion that has a conjugate acid having a pKₐ of 10 or higher.

2. A resin as claimed in claim 1, wherein the counterion is a monoanion.

3. A resin as claimed in claim 2, wherein the counterion is the hydroxide anion or an alkoxide anion.

4. A resin as claimed in claim 3, wherein the counterion is the methoxide or ethoxide anion.

5. A resin as claimed in claim 1, wherein the counterion of the at least one cationic methylated salt is an anion that has a conjugate acid having a PKₐ Of 11 or higher.

6. A resin as claimed in claim 1, wherein the counterion of the at least one cationic methylated salt is an anion that has a conjugate acid having a pKₐ of 11 to 18.

7. A resin as claimed in claim 1, wherein the at least one cationic methylated salt is immobilized on the solid support through a covalent bond.

8. A resin as claimed in claim 1, wherein the at least one cationic methylated salt is immobilized on the solid support through dipolar attraction.

9. A resin as claimed in claim 1, wherein the at least one cationic methylated salt is immobilized on the solid support through ion pairing.

10. A resin as claimed in claim 1, which comprises at least one cationic methylated sulfonium salt immobilized on the solid support.

11. A resin as claimed in claim 1, which comprises at least one cationic methylated sulfoxonium salt immobilized on the solid support.

12. A resin as claimed in claim 1, which comprises at least one cationic methylated selenonium salt immobilized on the solid support.

13. A resin as claimed in claim 1, wherein the at least one cationic methylated salt comprises a linker group between the sulfur or selenium atom of the salt and the solid support.

14. A resin as claimed in claim 13, wherein the linker group is an alkyl group, an aryl group, a heteroaryl group, or a combination thereof either linearly or branched,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof, and
wherein any methylene group, or each of two or more non-adjacent methylene groups, in an alkyl group may be replaced by an oxygen atom, and
wherein the sulfur and selenium atoms of the cationic group are bound to carbon atoms.

15. A resin as claimed in claim 14, wherein the electron withdrawing groups are chosen from halogen, nitro, cyano, hydroxyl, fluoroalkyl, perfluoroalkyl, nitrile, carboxyl, carboxylic ester, amide, sulfoxide, sulfone, carbonyl and ammonium groups.

16. A resin as claimed in claim 14, wherein the electron donating groups are chosen from alkoxy groups.

17. A resin as claimed in claim 16, wherein the electron donating groups are chosen from methoxy and ethoxy groups.

18. A resin as claimed in claim 1, wherein the solid support is a polymer.

19. A resin as claimed in claim 1, wherein the solid support is silica gel, alumina or diatomaceous earth.

20. A resin, comprising:
a solid support, and
at least one cationic methylated phosphonium salt immobilized on the solid support,
in which the counterion of the at least one cationic methylated salt is an anion that has a conjugate acid having a pKₐ of 10 or higher.

21. A resin as claimed in claim 1, which comprises at least one cationic methylated salt immobilized on the solid support as in formula (Ia), (Ib) or (Ic): 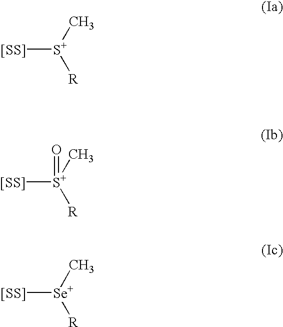
wherein
[SS] is the solid support; and
R is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and, and
wherein any methylene group, or each of two or more non-adjacent methylene groups, in an alkyl group in R may be replaced by an oxygen atom, and
wherein the indicated sulfur and selenium atoms are bound to carbon atoms.

22. A resin as claimed in claim 21, wherein the electron withdrawing groups are chosen from halogen, nitro, cyano, hydroxyl, fluoroalkyl, perfluoroalkyl, nitrile, carboxyl, carboxylic ester, amide, sulfoxide, sulfone, carbonyl and ammonium groups.

23. A resin as claimed in claim 21, wherein the electron donating groups are chosen from alkoxy groups.

24. A resin as claimed in claim 23, wherein the electron donating groups are chosen from methoxy and ethoxy groups.

25. A resin as claimed in claim 21, wherein R is —CH₃.

26. A resin as claimed in claim 1, which comprises at least one cationic methylated salt immobilized on the solid support as in formula (IIa), (IIb) or (IIc): 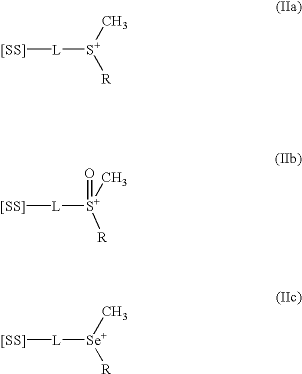
wherein
[SS] is the solid support and L is a linker group; and
L, R each independently of the other is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and
wherein any methylene group, or each of two or more non-adjacent methylene groups, in an alkyl group in L or R may be replaced by an oxygen atom, and
wherein the indicated sulfur and selenium atoms are bound to carbon atoms.

27. A resin as claimed in claim 26, wherein the electron withdrawing groups are chosen from halogen, nitro, cyano, hydroxyl, fluoroalkyl, perfluoroalkyl, nitrile, carboxyl, carboxylic ester, amide, sulfoxide, sulfone, carbonyl and ammonium groups.

28. A resin as claimed in claim 26, wherein the electron donating groups are chosen from alkoxy groups.

29. A resin as claimed in claim 28, wherein the electron donating groups are chosen from methoxy and ethoxy groups.

30. A resin as claimed in claim 26, wherein L and R each independently of the other is an alkyl group, which is unsubstituted or substituted as recited in claim 26.

31. A resin as claimed in claim 30, wherein R is —CH₃.

32. A resin as claimed in claim 26, wherein L and R are each independently the other an aryl or heteroaryl group, in which the aryl and heteroaryl groups are unsubstituted or substituted as recited in claim 26.

33. A resin as claimed in claim 26, wherein L is an alkyl group and R is an aryl or heteroaryl group, in which the alkyl, aryl and heteroaryl groups are unsubstituted or substituted as recited in claim 26.

34. A resin as claimed in claim 26, wherein L is an aryl or heteroaryl group and R is an alkyl group, in which the alkyl, aryl and heteroaryl groups are unsubstituted or substituted as recited in claim 26.

35. A resin as claimed in claim 26, wherein L is an aryl group, which is unsubstituted or substituted as recited in claim 26, and R is —CH₃.

36. A resin as claimed in claim 20, which comprises at least one cationic methylated salt immobilized on the solid support as in formula (lid): 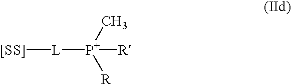
wherein
[SS] is the solid support and L is a linker group; and
L, R, R′ each independently of the other is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and
wherein any methylene group, or each of two or more non-adjacent methylene groups, in an alkyl group in L, R and R′ may be replaced by an oxygen atom, and
wherein the indicated phosphorous atom is bound to carbon atoms.

37. A resin as claimed in claim 36, wherein R and R′ are both —CH₃.

38. A resin as claimed in claim 36, wherein at least one of R and R′ is an aryl or heteroaryl group, which is unsubstituted or substituted as recited in claim 36.

39. A resin as claimed in claim 1, which comprises at least one cationic methylated salt immobilized on the solid support as in formula (IIa) or (IIIb): 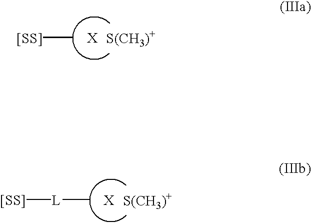
wherein
[SS] is the solid support and L is a linker group;
L is an alkyl, aryl or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and, and
X is a 5 to 10 membered aromatic or non-aromatic ring that may contain, in addition to the —S(CH₃)⁺ group, one or more atoms chosen from S, N and O,
which is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and
wherein any methylene group, or each of two or more non-adjacent methylene groups, in an alkyl group in L or X may be replaced by an oxygen atom, and
wherein the indicated sulfur atom is bound to carbon atoms.

40. A resin as claimed in claim 39, wherein the electron withdrawing groups are chosen from halogen, nitro, cyano, hydroxyl, fluoroalkyl, perfluoroalkyl, nitrile, carboxyl, carboxylic ester, amide, sulfoxide, sulfone, carbonyl and ammonium groups.

41. A resin as claimed in claim 39, wherein the electron donating groups are chosen from alkoxy groups.

42. A resin as claimed in claim 41, wherein the electron donating groups are chosen from methoxy and ethoxy groups.

43. A resin as claimed in claim 1, which comprises at least one cationic methylated salt immobilized on the solid support as in formula (IVa) or (IVb) 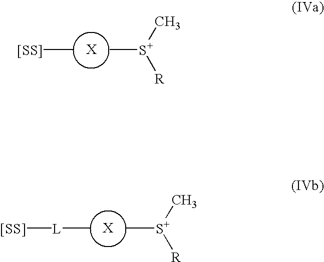
wherein
[SS] is the solid support and L is a linker group;
L, R each independently of the other is an alkyl, aryl or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and
X is a 5 to 1 0 membered non-aromatic carbocyclic or heterocyclic group,
which is unsubstituted or substituted by one or more further alkyl, aryl or heteroaryl groups, and
in which any alkyl, aryl and heteroaryl group may be unsubstituted or substituted by one or more further alkyl groups, one or more electron withdrawing groups, one or more electron donating groups, or a combination thereof; and,
wherein any methylene group, or each of two or more non-adjacent methylene groups, in an alkyl group in L, R or X may be replaced by an oxygen atom, and
wherein the indicated sulfur atom is bound to carbon atoms.

44. A resin as claimed in claim 43, wherein the electron withdrawing groups are chosen from halogen, nitro, cyano, hydroxyl, fluoroalkyl, perfluoroalkyl, nitrile, carboxyl, carboxylic ester, amide, sulfoxide, sulfone, carbonyl and ammonium groups.

45. A resin as claimed in claim 43, wherein the electron donating groups are chosen from alkoxy groups.

46. A resin as claimed in claim 45, wherein the electron donating groups are chosen from methoxy and ethoxy groups.

47. A resin, comprising:
a solid support, and
at least one cationic methylated sulfonium, sulfoxonium or selenonium salt immobilized on the solid support,
in which the counterion of the at least one cationic methylated salt is an anion that has a conjugate acid having a pKₐ of 10 or higher,
wherein the at least one cationic methylated salt is immobilized on the solid support as in formula (Va), (Vb) or (Vc): 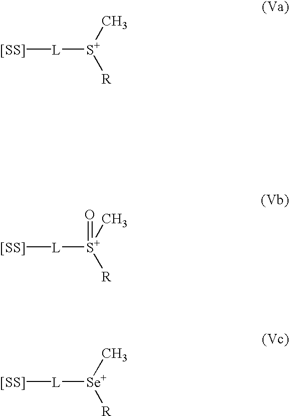
wherein
[SS] is the solid support and L is a linker group; and
L, R each independently of the other is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,
wherein any methylene group, or each of two or more non-adjacent methylene groups, in an alkyl group in L or R may be replaced by an oxygen atom, and
wherein the indicated sulfur and selenium atoms are bound to carbon atoms.

48. A resin as claimed in claim 47, wherein L is unsubstituted or substituted phenyl and R is —CH₃.

49. A resin as claimed in claim 47, wherein the counterion is the hydroxide ion or an alkoxide ion.

50. A resin as claimed in claim 49, wherein the counterion is a methoxide or ethoxide ion.

51. A resin, comprising:
a solid support, and
at least one cationic methylated sulfonium salt immobilized on the solid support,
in which the counterion of the at least one cationic methylated salt is an anion that has a conjugate acid having a pKₐ of 10 or higher,
wherein the at least one cationic methylated salt is immobilized on the solid support as in formula (VI): 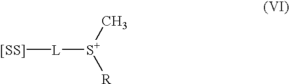
wherein
[SS] is the solid support and L is a linker group; and
L, R each independently of the other is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,
wherein any methylene group, or each of two or more non-adjacent methylene groups, in an alkyl group in L or R may be replaced by an oxygen atom, and
wherein the indicated sulfur atom is bound to carbon atoms.

52. A resin as claimed in claim 51, wherein L is unsubstituted or substituted phenyl and R is-CH₃.

53. A resin as claimed in claim 51, wherein the counterion is the hydroxide ion or an alkoxide ion.

54. A process for methylating the phenolic moiety of a compound, which comprises:
loading the compound onto a resin as claimed in claim 1, and
heating the loaded resin in a solvent under conditions sufficient to methylate the phenolic moiety of the compound.

55. A process for methylating the phenolic moiety of a compound, which comprises:
loading the compound onto a resin as claimed in claim 10, and
heating the loaded resin in a solvent under conditions sufficient to methylate the phenolic moiety of the compound.

56. A process for methylating the phenolic moiety of a compound, which comprises:
loading the compound onto a resin as claimed in claim 20, and
heating the loaded resin in a solvent under conditions sufficient to methylate the phenolic moiety of the compound.

57. A process for methylating the phenolic moiety of a compound, which comprises:
loading the compound onto a resin as claimed in claim 26, and
heating the loaded resin in a solvent under conditions sufficient to methylate the phenolic moiety of the compound.

58. A process as claimed in claim 54, which comprises methylating the phenolic moiety of morphine to form codeine.

59. A process as claimed in claim 55, which comprises methylating the phenolic moiety of morphine to form codeine.

60. A process as claimed in claim 56, which comprises methylating the phenolic moiety of morphine to form codeine.

61. A process as claimed in claim 57, which comprises methylating the phenolic moiety of morphine to form codeine.

62. A process as claimed in claim 54, which comprises loading the resin in presence of a solvent, and heating the loaded resin in the presence of a solvent which is the same as that used during the loading.

63. A process as claimed in claim 54, which comprises loading the resin in presence of a solvent, and heating the loaded resin in the presence of a solvent different from that used during the loading.

64. A process as claimed in claim 54, which comprises loading the compound onto the resin in the present of a solvent that comprises water.

65. A process as claimed in claim 54, which comprises loading the compound onto the resin in the presence of a solvent that comprises at least one alkanol.

66. A process as claimed in claim 65, which comprises loading the compound onto the resin in the presence of a solvent that comprises methanol, ethanol, or both methanol and ethanol.

67. A process as claimed in claim 54, which comprises loading the compound onto the resin in the presence of at least one ketonic solvent.

68. A process as claimed in claim 67, which comprises loading the compound onto the resin in the presence of at least one of acetone and butanone.

69. A process as claimed in claim 54, which comprises loading the compound onto the resin in the presence of tetrahydrofuran.

70. A process as claimed in claim 54, which comprises heating the loaded resin in the presence of a solvent that comprises at least one alkanol.

71. A process as claimed in claim 70, which comprises heating the loaded resin in the presence of a solvent that comprises methanol, ethanol, or both methanol and ethanol.

72. A process as claimed in claim 54, which comprises heating the loaded resin in the presence of a solvent that comprises an aromatic hydrocarbon.

73. A process as claimed in claim 72, which comprises heating the loaded resin in the presence of a solvent that comprises benzene or toluene.

74. A process as claimed in claim 54, which comprises heating the loaded resin in the presence of a solvent that comprises a non-aromatic hydrocarbon.

75. A process as claimed in claim 74, which comprises heating the loaded resin in the presence of a solvent that comprises cyclohexane.

76. A process as claimed in claim 54, which comprises loading the compound onto the resin in the presence of a solvent that comprises at least one alkanol, and heating the loaded resin in the present of a solvent that comprises at least one alkanol.

77. A process as claimed in claim 76, which comprises loading the compound onto the resin in the presence of a solvent that comprises methanol, ethanol, or both methanol and ethanol, and heating the loaded resin in the presence of a solvent that comprises methanol, ethanol, or both methanol and ethanol.

78. A process as claimed in claim 54, which comprises loading the compound onto the resin in the presence of a solvent that comprises methanol, ethanol, or both methanol and ethanol, and heating the loaded resin in the presence of a solvent that comprises toluene.

79. A process as claimed in claim 54, which further comprises methylating the expended resin and converting it to its hydroxide or alkoxide form in the presence of water, methanol, ethanol, or combinations thereof.

80. A process for methylating the phenolic moiety of morphine to form codeine, which comprises:
a) loading morphine onto a resin which comprises
a solid support, and
at least one cationic methylated sulfonium salt immobilized on the solid support,
in which the counterion of the at least one cationic methylated salt is an anion that has a conjugate acid having a pKₐ of 11 or higher,
wherein the at least one cationic methylated salt is immobilized on the solid support as in formula (VI): 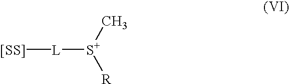
wherein
[SS] is the solid support and L is a linker group; and
L, R each independently of the other is an alkyl, aryl, or heteroaryl group,
in which the alkyl, aryl and heteroaryl group is unsubstituted or substituted by one or more further alkyl, aryl or heteroaryl groups, and
in which any alkyl, aryl and heteroaryl group may be unsubstituted or substituted by one or more further alkyl groups, or by one or more substituents chosen from halogen, nitro, cyano, hydroxyl, fluoroalkyl, perfluoroalkyl, nitrile, carboxyl, carboxylic ester, amide, sulfoxide, sulfone, carbonyl and ammonium, or by a combination thereof,
wherein any methylene group, or each of two or more non-adjacent methylene groups, in an alkyl group in L or R may be replaced by an oxygen atom, and
wherein the indicated sulfur atom is bound to carbon atoms, in the presence of a solvent that comprises water, one or more alkanols, or water and one or more alkanols, and
b) heating the loaded resin in a solvent that comprises one or more alkanols under conditions sufficient to methylate the phenolic moiety of the compound to form codeine.

81. A process as claimed in claim 80, which comprises loading the morphine onto the resin in the presence of a solvent that comprises of water, methanol, ethanol, or a combination thereof, and heating the loaded resin in a solvent that comprises methanol, ethanol, or a combination thereof.

82. A process as claimed in claim 80, which comprises heating the loaded resin at a temperature of 60-65° C. for a period of one to fifteen hours.

83. A process as claimed in claim 80, which comprises heating the loaded resin at a temperature of 60-65° C. for a period of ten to fifteen hours.

84. A process for converting a carboxylic acid moiety of a compound to its methyl ester, which comprises:
loading the compound onto a resin comprising:
a solid support, and
at least one cationic methylated sulfonium, sulfoxonium, selenonium or phosphonium salt immobilized on the solid support,
in which the counterion of the at least one cationic methylated salt is an anion that has a conjugate acid having a pKₐ of 7 or higher, and
heating the loaded resin in a solvent under conditions sufficient to form a methyl ester of the carboxylic acid moiety.

85. A process for methylating a thiol compound, which comprises:
loading the compound onto a resin comprising:
a solid support, and
at least one cationic methylated sulfonium, sulfoxonium, selenonium or phosphonium salt immobilized on the solid support,
in which the counterion of the at least one cationic methylated salt is an anion that has a conjugate acid having a pKₐ of 10 or higher, and
heating the loaded resin in the solvent under conditions sufficient to methylate the thiol compound.

86. A resin, comprising:
a solid support, and
at least one cationic alkylated sulfonium, sulfoxonium, selenonium or phosphonium salt of 2 to 8 carbon atoms immobilized on the solid support,
in which the counterion of the at least one cationic alkylated salt is an anion that has a conjugate acid having a pKₐ of 10 or higher.

87. A process for alkylating the phenolic moiety of a compound, or for alkylating a thiol compound, which comprises:
loading the compound onto a resin as claimed in claim 86, and
heating the loaded resin in a solvent under conditions sufficient to alkylate the compound with a C₂-C₈ alkyl.

88. A process for converting a carboxylic acid moiety of a compound to its alkyl ester, which comprises:
loading the compound onto a resin comprising:
a solid support, and
at least one cationic alkylated sulfonium, sulfoxonium, selenonium or phosphonium salt of 2 to 8 carbon atoms immobilized on the solid support,
in which the counterion of the at least one cationic alkylated salt is an anion that has a conjugate acid having a pKₐ of 7 or higher, and
heating the loaded resin in a solvent under conditions sufficient to form a C₂-C₈ alkyl ester of the compound.

89. A resin, comprising:
a solid support, and
at least one cationic benzylated sulfonium, sulfoxonium, selenonium or phosphonium salt immobilized on the solid support,
in which the counterion of the at least one cationic benzylated salt is an anion that has a conjugate acid having a pKₐ of 10 or higher,
wherein the benzyl group is unsubstituted or substituted by one or more alkyl groups, one or more electron withdrawing groups, one or more electron donating groups, or a combination thereof.

90. A process for benzylating the phenolic moiety of a compound, or of benzylating a thiol compound, which comprises:
loading the compound onto a resin as claimed in claim 89, and
heating the loaded resin in a solvent under conditions sufficient to benzylate the compound.

91. A process for converting a carboxylic acid moiety of a compound to its benzyl ester, which comprises:
loading the compound onto a resin comprising:
a solid support, and
at least one cationic benzylated sulfonium, sulfoxonium, selenonium or phosphonium salt immobilized on the solid support,
in which the counterion of the at least one cationic benzylated salt is an anion that has a conjugate acid having a pKₐ of 7 or higher, and,
wherein the benzyl group is unsubstituted or substituted by one or more alkyl groups, one or more electron withdrawing groups, one or more electron donating groups, or a combination thereof, and
heating the loaded resin in a solvent under conditions sufficient to form a benzyl ester of the compound.

92. A resin, comprising:
a solid support, and
at least one cationic allylated sulfonium, sulfoxonium, selenonium or phosphonium salt immobilized on the solid support,
in which the counterion of the at least one cationic allylated salt is an anion that has a conjugate acid having a pKₐ of 10 or higher.

93. A process for allylating the phenolic moiety of a compound, or of allylating a thiol compound, which comprises:
loading the compound onto a resin as claimed in claim 92, and
heating the loaded resin in a solvent under conditions sufficient to allylating the compound.

94. A process for converting a carboxylic acid moiety of a compound to its allyl ester, which comprises:
loading the compound onto a resin comprising:
a solid support, and
at least one cationic allylated sulfonium, sulfoxonium, selenonium or phosphonium salt immobilized on the solid support,
in which the counterion of the at least one cationic allylated salt is an anion that has a conjugate acid having a pKₐ of 7 or higher, and
heating the loaded resin in a solvent under conditions sufficient to form an allyl ester of the compound.
